(19) United States
(12) Patent Application Publication
(10) Pub. No.: US 2008/0288019 A1
(43) Pub. Date: Nov. 20, 2008

(54) Electrochemical management of pain

(76) Inventor: Adam Heller, Austin, TX (US)

Correspondence Address: CLARK & ELBING LLP, 101 FEDERAL STREET, BOSTON, MA, 02110, US

(21) Appl. No.: 12/012,116

(22) Filed: Jan. 31, 2008

Related U.S. Application Data
(60) Provisional application No. 60/887,431, filed on Jan. 31, 2007.
(60) Provisional application No. 60/930,261, filed on May 15, 2007.

(51) Int. Cl. A61N 1/04

(52) U.S. Cl. 607/46

(57) ABSTRACT

The invention features electrochemical methods and devices for the treatment of pain.

### CROSS-REFERENCE TO RELATED APPLICATIONS

This application claims benefit of U.S. Provisional Ser. No. 60/887,431 filed on Jan. 31, 2007, and U.S. Provisional Ser. No. 60/930,261, filed on May 15, 2007, each of which is incorporated herein by reference.

### BACKGROUND OF THE INVENTION

Chronic pain is one of the most important clinical problems in all of medicine. For example, it is estimated that over 5 million people in the United States are disabled by back pain. The economic cost of chronic back pain is enormous, resulting in over 100 million lost work days annually at an estimated cost of $50-100 billion. It has been reported that approximately 8 million people in the U.S. report that they experience chronic neck or facial pain and spend an estimated $2 billion a year for treatment. The cost of managing pain for oncology patients is thought to approach $12 billion. Chronic pain disables more people than cancer or heart disease and costs the American public more than both cancer and heart disease combined. In addition to the physical consequences, chronic pain has numerous other costs including loss of employment, marital discord, depression and prescription drug addiction. It goes without saying, therefore, that reducing the morbidity and costs associated with persistent pain remains a significant challenge for the healthcare system.

Intractable severe pain resulting from injury, illness, scoliosis, spinal disc degeneration, spinal cord injury, malignancy, arachnoiditis, chronic disease, pain syndromes (e.g., failed back syndrome, complex regional pain syndrome) and other causes is a debilitating and common medical problem. In many patients, the continued use of analgesics, particularly drugs like narcotics, are not a viable solution due to tolerance, loss of effectiveness, and addiction potential. In an effort to combat this, neurostimulation devices have been developed to treat severe intractable pain that is resistant to other traditional treatment modalities such as drug therapy, invasive therapy (surgery), or behavioral/lifestyle changes.

It has been reported that neurostimulation works by delivering low voltage electrical stimulation to the spinal cord or a particular peripheral nerve in order to block the sensation of pain. The Gate Control Theory of Pain (Ronald Melzack and Patrick Wall) hypothesizes that there is a “gate” in the dorsal horn of the spinal cord that controls the flow of pain signals from the peripheral receptors to the brain. It is speculated that the body can inhibit the pain signals (“close the gate”) by activating other (non-pain) fibers in the region of the dorsal horn. Neurostimulation devices are implanted in the epidural space of the spinal cord to stimulate non-noxious nerve fibers in the dorsal horn and mask the sensation of pain. As a result the patient typically experiences a tingling sensation (known as paresthesia) instead of pain. With neurostimulation, the majority of patients will report improved pain relief (50% reduction), increased activity levels, and a reduction in the use of narcotics.

What is needed are more and better treatment options. It is an object of the invention to provide new methods and devices for the treatment of pain and itch.

### SUMMARY OF THE INVENTION

Applicants have discovered that neurostimulation methods and devices configured to promote the electrochemical generation of oxidants (i.e., reconfigured as an electrolytic device) are useful for the treatment of pain and itch.

Accordingly, in a first aspect, the invention features an implantable medical device for the treatment of pain including a DC power supply in electrical communication with a first electrode and a second electrode, wherein the first electrode includes an oxidation catalyst and the second electrode includes either no catalyst, a reduction catalyst, or a reducible metal salt or metal oxide.

In another aspect the invention features an implantable medical device for the treatment of pain including a DC power supply in electrical communication with a first electrode and a second electrode, wherein the first electrode includes either no catalyst or an oxidation catalyst and the second electrode includes a reduction catalyst or a reducible metal salt or metal oxide.

In certain embodiments of the above aspects, the first electrode and the second electrode are separated by an ion conducting membrane (e.g., a charge mosaic membrane). In certain embodiments, the ion conducting membrane can conduct both anions and cations.

In a related aspect, the invention features an implantable medical device for the treatment of pain including a DC power supply in electrical communication with a first electrode and a second electrode, wherein the first electrode and the second electrode are separated by an ion conducting membrane (e.g., a charge mosaic membrane). In some embodiments, the ion conducting membrane can conduct both anions and cations.

In certain embodiments of the above aspects, the first electrode includes an oxidation catalyst and the second electrode includes a reduction catalyst or a reducible metal salt or metal oxide. For example, the first electrode can include an oxidation catalyst for catalyzing the electrooxidation of chloride anion, the second electrode can include a reducible metal salt or metal oxide, and/or the electrode can include a reduction catalyst.

The invention further features an implantable medical device for the treatment of pain including an AC power supply in electrical communication with a first electrode and a second electrode, wherein each of the first electrode and the second electrode include an oxidation catalyst.

In still other embodiments of the above aspects, the implantable medical device when implanted in a patient generates hypochlorous acid in an amount sufficient to treat pain.

In any of the above aspects, the implantable medical device can further include a reservoir in fluid communication with the first electrode, a nerve, or proximate to a first electrode or nerve (e.g., less than 1 cm, 0.5 cm, 0.2 cm, or 0.1 cm from the electrode or treated nerve), the reservoir including an oxidizable agent selected from amines, amides, thiols, and salts thereof. The oxidizable agent can be any oxidizable agent described herein. In certain embodiments, the reservoir further includes a chloride salt. For example, the reservoir can include about an isotonic amount of chloride (e.g., 0.15 M), while the oxidizable agent is present in the reservoir at a concentration of less than 0.05 M, 0.03 M, 0.015 M, or 0.005 M. Desirably, the chloride concentration in the reservoir is from 0.5 to 0.01 M, 0.3 to 0.05 M, or 0.2 to 0.1 M, while the concentration of oxidizable agent in the reservoir is from 0.1 to 0.005 M, 0.075 to 0.01 M, or 0.05 to 0.015 M.

The invention further features an implantable medical device for the treatment of pain including (i) a power supply in electrical communication with a first electrode and a second electrode, wherein an oxidant is electrochemically generated at least at the first electrode; (ii) a reservoir including a solution of an oxidizable agent in fluid communication with the electrochemically generated oxidant, wherein the oxidant oxidizes the oxidizable agent to produce an oxidized agent; and (iii) an exit port in fluid communication with the oxidized agent, wherein after implantation the implantable medical device generates oxidized agent in an amount sufficient to treat pain. The oxidizable agent can be selected from amines, amides, thiols, and salts thereof. In certain embodiments, the oxidizable agent is selected from ammonia, taurine, glutathione, glutathione sulfonamide, and salts thereof.

For any of the devices of the invention including a reservoir, the reservoir can be configured to be completely implanted within the patient. Alternatively, the reservoir can be configured to be positioned external to the patient and further including a cannula in fluid communication with the oxidant and an exit port, wherein the exit port is implanted within the patient. The reservoir may also be configured to deposit its contents in proximity to an electrode or a nerve being treated. In certain embodiments, the reservoir is configured to sit on the skin of the subject. In still other embodiments, the reservoir is refillable. In certain embodiments, the reservoir further includes a chloride salt. For example, the reservoir can include about an isotonic amount of chloride (e.g., 0.15 M), while the oxidizable agent is present in the reservoir at a concentration of less than 0.05 M, 0.03 M, 0.015 M, or 0.005 M. Desirably, the chloride concentration in the reservoir is from 0.5 to 0.01 M, 0.3 to 0.05 M, or 0.2 to 0.1 M, while the concentration of oxidizable agent in the reservoir is from 0.1 to 0.005 M, 0.075 to 0.01 M, or 0.05 to 0.015 M.

In a related aspect, the invention features a method of treating pain in a patient in need thereof by (i) implanting an electrolytic device of the invention in the patient at the site of pain and (ii) operating the device to generate an oxidant in an amount sufficient to treat the pain. The pain to be treated can be, for example, nociceptive pain, somatic pain, visceral pain, procedural pain, or inflammatory pain caused by trauma or surgery. In certain embodiments, the pain is caused by trauma, surgery, herniation of an intervertebral disk, spinal cord injury, shingles, HIV/AIDS, cancer related pain, amputation, carpal tunnel syndrome, diabetic neuropathy, postherpetic neuralgia, or a musculoskeletal disorder.

Oxidation catalysts which can be used in accordance with the methods and devices of the invention include, without limitation, an oxide of ruthenium (e.g., ruthenium dioxide) or an oxide of iridium (e.g., iridium dioxide). These can be coated, for example, on an underlayer of an oxide of titanium (e.g., titanium dioxide), ruthenium dioxide, or an oxide of tantalum (e.g., Ta₂O₅) on the respective metals, Ti or Ta, or their alloys.

Reducible metal salt or metal oxides which can be used in accordance with the methods and devices of the invention include, without limitation, silver salts (e.g., silver chloride) or nickel oxide.

Reduction catalysts which can be used in accordance with the methods and devices of the invention include, without limitation, platinum, palladium, silver, gold, copper, a porphyrin-metal complex, a phthalocyanin-metal complex, a polyoxometalate of molybdenum, a polyoxometalate of tungsten, a quinone, or a multicopper oxidase enzyme.

Unless otherwise designated, in any of the above methods and devices, the power supply can be a DC power supply or an AC power supply. When an AC power supply is used, the power supply desirably has a frequency of less than 100 Hz, 10 Hz, 10 Hz, 1 Hz, or 0.1 Hz. When a DC power supply is used, in certain embodiments the first electrode is an anode comprising graphite.

In any of the above methods and devices, the power supply can operate at a voltage from 0.6 V to 5.0 V, preferably from 0.6 V to 4.0 V, 0.6 V to 3.0 V, 0.6 V to 2.0 V, or 0.6 V to 1.5 V between the electrodes.

In any of the above methods and devices, the power supply desirably operates at a current density of less than 5 mA/cm², 4 mA/cm², 3 mA/cm², 2 mA/cm², 1 mA/cm², 0.5 mA/cm², 0.3 mA/cm², or even 0.2 mA/cm².

The invention also features a biocompatible implantable matrix including (i) glucose oxidase or lactate oxidase; and (ii) myeloperoxidase. In certain embodiments, the matrix is a hydrogel. The biocompatible implantable matrix can further include an oxidizable agent is selected from amines, amides, thiols, and salts thereof. The oxidizable agent can be any oxidizable agent described herein.

In a related aspect, the invention features a method of treating pain in a patient in need thereof by implanting into the patient a biocompatible implantable matrix of the invention. In certain embodiments, the biocompatible implantable matrix is implanted at the site of pain.

The term “pain” is used herein in the broadest sense and refers to all types of pain, including acute and chronic pain, such as nociceptive pain, e.g. somatic pain and visceral pain; inflammatory pain, dysfunctional pain, idiopathic pain, neuropathic pain, e.g., peripherally generated pain and cancer pain.

The term “nociceptive pain” is used to include all pain caused by noxious stimuli that threaten to or actually injure body tissues, including, without limitation, by a cut, bruise, bone fracture, crush injury, and the like. Pain receptors for tissue injury (nociceptors) are located mostly in the skin, musculoskeletal system, or internal organs. The term “nociceptive pain”

The term “somatic pain” is used to refer to pain arising from bone, joint, muscle, skin, or connective tissue. This type of pain is typically well localized.

The term “visceral pain” is used herein to refer to pain arising from visceral organs, such as the respiratory, gastrointestinal tract and pancreas, the urinary tract and reproductive organs. Visceral pain includes pain caused by tumor involvement of the organ capsule. Another type of visceral pain, which is typically caused by obstruction of hollow viscus, is characterized by intermittent cramping and poorly localized pain. Visceral pain may be associated with inflammation as in cystitis or reflux esophagitis.

The term “inflammatory pain” includes pain associated with active inflammation that may be caused by trauma, surgery, or infection.

The term “neuropathic pain” is used herein to refer to pain originating from abnormal processing of sensory input by the peripheral nervous system consequent on a lesion to this system.

The term “procedural pain” refers to pain arising from a medical, dental or surgical procedure wherein the procedure is usually planned or associated with acute trauma.

The term “itch” (also known as pruritus) is used herein in the broadest sense and refers to all types of itching and stinging sensations localized and generalized, acute intermittent and persistent. The itch may be idiopathic, allergic, metabolic, drug-induced, due to liver, kidney disease, or cancer.

By “patient” is meant any animal. In one embodiment, the patient is a human. Other animals that can be treated using the methods and devices of the invention include but are not limited to non-human primates (e.g., monkeys, gorillas, chimpaneees), domesticated animals (e.g., horses, pigs, goats, rabbits, sheep, cattle, llamas), companion animals (e.g., guinea pigs, rats, mice, lizards, snakes, dogs, cats, fish, hamsters, and birds), animals participating in races or contests (horses, camels, dogs, birds), and marine mammals.

The term “salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the agents of the invention, or separately by reacting the free base function with a suitable organic acid. Representative acid addition salts include but are not limited to acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, taurate, thiocyanate, toluenesulfonate, undecanoate, valerate salts, and the like. Representative alkali or alkaline earth metal salts include but are not limited to sodium, lithium, potassium, calcium, magnesium, and the like, as well as nontoxic ammonium (e.g., primary ammonium salts, such as methylammonium and ethylammonium, and secondary ammonium salts, such as dimethylammonium and diethylammonium), quaternary ammonium (e.g., tetramethylammonium, tetraethylammonium), and the like.

By “treating pain” is meant preventing, reducing, or eliminating the sensation of pain in a subject. To treat pain, according to the methods of this invention, the treatment does not necessarily provide therapy for the underlying pathology that is causing the painful sensation. Treatment of pain can be purely symptomatic.

By “treating itch” is meant preventing, reducing, or eliminating the sensation of itch in a subject. To treat itch, according to the methods of this invention, the treatment does not necessarily provide therapy for the underlying pathology that is causing the itch. Treatment of itch can be purely symptomatic.

By “an amount sufficient” is meant an amount of an oxidant administered in a device or using a method of the invention required to prevent, reduce, or eliminate the sensation of pain (nociception) or itch. The effective amount of oxidant used to practice the present invention for therapeutic treatment of pain or itch varies depending upon the manner of administration, the age, and body weight, of the subject as well as the route of administration and underlying pathology that is causing the pain or itch. Ultimately, the attending physician or veterinarian will decide the appropriate amount and dosage regimen. Such amount is referred to as a “sufficient” amount.

By “musculoskeletal disorder” is meant an immune system-related disorder of the muscles, ligaments, bones, joints, cartilage, or other connective tissue. Among the most commonly-occurring musculoskeletal disorders are various forms of arthritis, e.g., osteoarthritis, rheumatoid arthritis, juvenile rheumatoid arthritis, and gout. Other musculoskeletal disorders include acquired hyperostosis syndrome, acromegaly, ankylosing spondylitis, Behcet's disease, bone diseases, bursitis, cartilage diseases, chronic fatigue syndrome, compartment syndromes, congenital hypothyroidism, congenital myopathies, dentigerous cyst, dermatomyositis, diffuse idiopathic skeletal hyperostosis, Dupuytren's contracture, eosinophilia-myalgia syndrome, fasciitis, Felty's syndrome, hallux valgus, infectious arthritis, joint diseases, Kabuki make-up syndrome, Legg-Perthes disease, lupus, Lyme disease, Melas syndrome, metabolic bone diseases, mitochondrial myopathies, mixed connective tissue disease, muscular diseases, muscular dystrophies, musculoskeletal abnormalities, musculoskeletal diseases, myositis, myositis ossificans, necrotizing fasciitis, neurogenic arthropathy, osteitis deformans, osteochondritis, osteomalacia, osteomyelitis, osteonecrosis, osteoporosis, Paget's disease, Pierre Robin syndrome, polymyalgia rheumatica, polymyositis, postpoliomyelitis syndrome, pseudogout, psoriatic arthritis, reactive arthritis, Reiter disease, relapsing polychondritis, renal osteodystrophy, rhabdomyolysis, rheumatic diseases, rheumatic fever, scleroderma, Sever's disease (calceneal apophysitis), Sjögren's syndrome, spinal diseases, spinal stenosis, Still's disease, synovitis, temporomandibular joint disorders, tendinopathy, tennis elbow, tenosynovitis, Tietze's syndrome, and Wegener's granulomatosis.

A used herein, “local” and “locally” refer to the passage of an electrical current at, or adjacent to, the site of pain or itching, or near part of a nerve transmitting the pain or itching-signal. For example, the pain-relieving oxidant or oxidant precursor generating electrodes of the electrolytic devices of the invention are typically within about 2 cm or less from the affected nerve ending, preferably within less than about 0.5 cm and most preferably within less than about 2 mm from the ending.

The term “electrolytic device” refers to an electrical device for producing pain relieving oxidants in vivo or in, or adjacent to, a reservoir for delivering a pain relieving oxidant to a subject. Such devices can be, for example, a neurostimulation device modified as described herein to safely increase the faradaic yield of oxidants, oxidants or their precursors, in close proximity to nervous tissue. The electrolytic device may be delivered to many different parts of the nervous system, including, spinal cord, peripheral nerves, muscles, and tissues. As such, electrolytic devices are designed to conform to the different anatomical structures and nervous system characteristics at the site of implantation. The current employed in an electrolytic device can be direct or alternating. When alternating, the frequency can be generally less than about 100 Hz, 10 Hz, 1 Hz, or 0.1 Hz.

“Lead” refers to an electrical connection formed of a conductor to carry electrical signals from the generator to a working electrode. Unlike in the medical usage of the term, it excludes the electrode itself. Typically, electrical leads are composed of a connector assembly and a lead body. The electrical lead may be a wire or other material that transmits electrical current from a power supply (e.g., a battery).

As used herein, “unipolar system” refers to one electrode placed in the treated volume element of the tissue, at least one other electrode being located outside this volume. “Multi-polar system,” such as bipolar, tripolar and quadripolar system refers to that multiple, such two, three or four electrodes are placed in the treated volume element of the tissue.

“Implanted” refers to having completely or partially placed a device within a host. A device is partially implanted when some of the device reaches, or extends, to the outside of a host.

As used herein, the term “charge mosaic membrane” refers to a membrane or other support that includes a plurality of charged groups, wherein some of the charged groups are positively charged (e.g., quaternary ammonium groups), wherein other groups are negatively charged (e.g., sulfonic acid groups), and wherein the plurality of charged groups are disposed in the membrane or other support such that selected cations and anions (e.g., H⁺ and OH⁻) can penetrate the membrane or other support while blocking or retarding the transport of solvent and/or other neutral species.

As used herein, “electrochemical synthesis” refers to the production of a pain-relieving oxidant using the methods and devices of the invention.

As used herein, the term “multicopper oxidase enzyme” refers to the multicopper oxidases characterized by the presence of four or more copper ions in either mono-or binuclear configuration. Multicopper oxidases are known to couple the one electron oxidation of a substrate to the four electron reduction of molecular oxygen to water (see, e.g., di Patti et al., Protein Eng 12:895 (1998)).

The implantable devices and materials of the invention can be placed proximal to the treated nerve, e.g., within less than 1 cm, 0.5 cm, or 0.2 cm from the treated nerve.

Other features and advantages of the invention will be apparent from the following detailed description and the claims.

### DETAILED DESCRIPTION

We have discovered that neurostimulation methods and devices when modified to promote the electrochemical generation of oxidants are useful for the treatment of pain and itch. The present invention provides neurostimulator leads and/or electrodes that are coated with electrocatalysts and/or electrochemically oxidizable agents. Such coatings can enhance pain relief during neurostimulation and are described in more detail below. The present invention also provides implanted electrochemical cells and membranes arranged to avoid excessive acidification near the electrocatalyst-coated oxidant generating electrodes.

### Implantable Electrolytic Devices

Pain management neurostimulation systems consist of a power supply that provides electrical power, at least two wires or leads, each connected to at least one electrode. At least one electrode electrically stimulates the spinal cord or targeted nerve. The leads are connected to the electrodes by either permanent, for example soldered, contacts or by separable contacts. They are also connected to the power source by either permanent, for example soldered contacts, or by separable contacts. The neurostimulation systems known in the art can be modified, as provided herein, to produce an electrolytic device that generates oxidants for pain relief.

The current passed can be a DC current, an AC current, or any combination of the two, e.g. a DC current with an AC component. When a DC current is passed, or when a current having a DC component, is passed, at least one oxidant, or oxidant precursor, is generated at one ore more electrodes, termed here first electrode(s) or anode(s). At another electrode or group of electrodes, termed here second electrode(s) or cathode(s), a chemical is reduced. When an AC current is passed, at least one oxidant, or oxidant precursor, is generated at one ore more electrodes during the half cycle in which electrons flow into the electrode from a solution species that is being oxidized, termed here “anodic half cycle” or “oxidizing half cycle”. In the next half cycle a solution species, that may be, for example, the just oxidized species, water itself, or dissolved oxygen is reduced by electrons flowing in the electron to the solution species being reduced.

In accordance with the present invention, it is the oxidant generated at the anode (or in AC operation in the anodic half-cycle), or its oxidant daughter product, that relieves pain. In one aspect, a DC current is used to generate the oxidant, or its precursor. Although the pain can be relieved by the oxidant generated at the anode, it is usually relieved by its reaction products, which are oxidizing daughter products formed in subsequent chemical oxidation reactions of the electrochemically generated oxidant.

The advantage of passing a DC current is that the yield of the pain relieving oxidant and/or its precursor oxidant is generally high. It can be particularly high when the anode and the cathode are well-separated. The yield of the oxidant can be particularly high when the electrocatalyst of the anode catalyzes the rapid electrooxidation of chloride anion and the electrocatalyst of the cathode catalyzes the rapid electroreduction of water and/or of dissolved oxygen. The disadvantage of passing a DC current is that an acid, requiring neutralization, can be co-generated with the oxidant at the anode and a base can be generated at the cathode. The acid or base, if strong, as it is when the current density is high, for example about 50 mA/cm², but not when it is low, for example less than about 1 mA/cm², can be cytotoxic and may require provision for neutralization. The acid generated at the anode can be neutralized by base generated at the cathode while maintaining a high faradaic yield by making the distance between the anode (or anodes) and the cathode (or cathodes) small and inserting a membrane preferably permeating the positively charged ions and/or negatively charged ions of ionically dissociated strong acids and/or bases, but not permeating rapidly non-ionic or zwitterionic oxidation products.

The advantage of passing an AC current is that the acid generated at an electrode in the oxidizing half cycle is neutralized by the base generated in the reducing half cycle. Its disadvantage is that the faradaic yield of the electrochemically generated oxidant can be low because the oxidant generated in the oxidizing half cycle can be reduced in the reducing half cycle. Hence it is advantageous to provide for avoiding the efficient reduction of the oxidant. The faradaic yield of the oxidant produced when an AC current is passed can be increased by rapidly reacting the oxidant produced in the oxidizing half-cycle with a controllably released agent or with an injected or infused agent to form the pain relieving compound or its precursor, some or all of the oxidant generated in the oxidizing half cycle reacting with the agent before the direction of the current is reversed. It is generally advantageous to operate the system at a low AC frequency, so as to provide enough time for the oxidant to oxidize with the controllably released, infused or injected agent, or in absence, with oxidant producing tissue components, such as glutathione (GSH) oxidized to oxidized glutathione (GSSG); or glutathione oxidized to glutathione sulfonamide (GSA), which is further oxidized to N-chlorogutathione sulfonamide, where the sulfonamide nitrogen is chlorinated; or taurine, oxidized to N-chlorotaurine, all of which are known to by synthesized in the body. Typically, the preferred AC frequency is less than 10 Hz, 1 Hz, or even 0.1 Hz.

The electrolytic devices of the invention can be battery powered, radio-frequency (RF) powered, or a combination of both. RF includes all frequencies for which receiving and/or transmitting antennas of conveniently wearable lengths are available. In general, there are two types of electrolytic devices: those that are surgically implanted and are completely internal (i.e., the battery, leads, and electrodes are implanted), and those with internal (electrodes, leads, and radio-frequency receiver) and external (power source and antenna) components. For internal, battery-powered electrolytic devices, an implanted, non-rechargeable battery and the leads and electrodes are all surgically implanted. The settings of the totally implanted electrolytic device may be controlled by the host by using an external magnet and the implant has a lifespan of two to four years. For radio-frequency powered electrolytic devices, the radio-frequency is transmitted from an externally worn source to an implanted passive receiver which charges a rechargeable battery. The radio-frequency powered system provides greater power and can provide power to multiple electrodes and/or to larger electrodes and/or to electrodes generating at a higher rate the pain-alleviating oxidant, providing thereby a greater flux of the pain relieving oxidant to the treated nerve ending or other nerve-part. Optionally, the implanted battery can be recharged also by connection to an external charger by a pair of fine wires passing through the skin, although in this case special feed-throughs are needed to avoid infection.

In general, the preferred usage of the electrolytic devices of this invention is in relieving pain signaled by the peripheral nervous system. The electrolytic devices of the invention can be made, however, by modifying an existing central nervous system neurostimulation device as provided herein. There are numerous neurostimulation devices that can be adapted for use as, for example, an electrolytic device of the invention, including, without limitation, neurostimulation devices designed for spinal cord stimulation in the management of pain control, postural positioning and other disorders. Examples of include those composed of a sensor that detects the position of the spine and a stimulator that automatically emits a series of pulses which decrease in amplitude when back is in a supine position (see, for example, U.S. Pat. Nos. 5,031,618 and 5,342,409, each of which is incorporated herein by reference). The electrolytic device may include electrodes and a control circuit which generates pulses and rest periods based on intervals corresponding to the body's activity and regeneration period as a treatment for pain (see, e.g., U.S. Pat. No. 5,354,320). The electrolytic device, which may be implanted within the epidural space parallel to the axis of the spinal cord, may transmit data to a receiver which generates a spinal cord stimulation pulse that may be delivered via a coupled, multi-electrode (see, e.g., U.S. Pat. No. 6,609,031). The electrolytic device may be a stimulation catheter lead with a sheath and at least three electrodes that provide pain relieving oxidants to neural tissue (see, e.g., U.S. Pat. No. 6,510,347). The electrolytic device may be a self-centering epidural spinal cord lead connected to an electrode with a pivoting region to stabilize the lead and electrode (see, e.g., U.S. Pat. No. 6,308,103). Neurostimulators that can be converted to electrolytic devices as described herein are described in U.S. Pat. Nos. 6,546,293; 6,236,892; 4,044,774 and 3,724,467, each of which is incorporated herein by reference.

Still other neurostimulators that can be converted to electrolytic devices as described herein are commercially available neurostimulation devices for the management of chronic pain and include the SYNERGY, INTREL, X-TREL, and MATTRIX neurostimulation systems from Medtronic, Inc. The percutaneous leads and electrodes in this system can be quadripolar (4 electrodes), such as the PISCES-QUAD, PISCES-QUAD PLUS and the PISCES-QUAD Compact, or octapolar (8 electrodes) such as the OCTAD lead-electrode system. The surgical leads themselves are quadripolar, such as the SPECIFY Lead-electrode system, the RESUME II Lead-electrode system, the RESUME TL Lead-electrode system and the ON-POINT PNS Lead-electrode system, to create multiple stimulation combinations and a broad area of paresthesia. These neurostimulation systems and associated lead-electrode systems are described in U.S. Pat. Nos. 6,671,544; 6,654,642; 6,360,750; 6,353,762; 6,058,331; 5,342,409; 5,031,618 and 4,044,774, each of which is incorporated herein by reference. Other commercially available systems that may useful for the practice of this invention as described herein include the rechargeable PRECISION Spinal Cord Stimulation System (Advanced Bionics Corporation, Sylmar, Calif.; which is a Boston Scientific Company) which can drive up to 16 electrodes (see e.g., U.S. Pat. Nos. 6,735,474; 6,735,475; 6,659,968; 6,622,048; 6,516,227 and 6,052,624); the GENESIS XP Spinal Cord Stimulator available from Advanced Neuromodulation Systems, Inc. (Plano, Tex.; see e.g., U.S. Pat. Nos. 6,748,276; 6,609,031 and 5,938,690); and the Vagus Nerve Stimulation (VNS) Therapy System available from Cyberonics, Inc. (Houston, Tex.; see e.g., U.S. Pat. Nos. 6,721,603 and 5,330,515).

Electrolytic devices may also be classified based on their source of power, which includes: battery powered, radio-frequency (RF) powered, or a combination of both types. For battery powered electrolytic devices, an implanted, non-rechargeable or RF-recharged battery is usually used as the source of power. The battery, an optional RF-receiving coil and the leads with their electrodes are all surgically implanted and thus the electrolytic device, other than the optional transmitting coil, is completely internal. The settings of the totally implanted electrolytic device can be controlled by the patient through an external magnet. The lifetime of the implant, when powered by a non-rechargeable battery, is generally limited by the duration of battery life and ranges from two to four years depending upon usage and power requirements. For RF-powered electrolytic devices, the radio-frequency is transmitted from an externally worn source to an implanted passive receiver, which charges usually an implanted rechargeable battery, but may optionally charge a capacitor, such as an electrochemical supercapacitor. Since the source of power for the transmitting coil can the grid, or a readily rechargeable battery, or a replaceable non-rechargeable battery, the radio-frequency system provides greater power and can power electrodes generating electrochemically a greater amount or flux of the pain-relieving oxidant or its precursor; or it can power a greater number of oxidant generating electrodes; or it can power electrodes having a greater area at which more oxidant is generated. Specific earlier disclosed examples include an electrolytic device that has a battery power source contained within to supply power over an eight hour period in which power may be replenished by an external radio frequency coupled device (see, for example, U.S. Pat. No. 5,807,397, incorporated herein by reference) or an electrolytic device which is controlled by an external transmitter using data signals and powered by radio frequency (see, for example, U.S. Pat. No. 6,061,596, incorporated herein by reference).

Faradaic Efficiency

The rate at which the pain-relieving oxidant, or its oxidant precursor, is produced in its electrochemical synthesis is determined by the faradaic efficiency of the electrosynthesis and by the current. In general, the dose-rate of the pain relieving-oxidant is between about 10⁻⁹ moles per hour and about 10⁻⁴ moles per hour. Because a charge of 2 Faradays is usually consumed in the electrochemical synthesis of 1 mole of oxidant or oxidant precursor at 100% faradaic efficiency. The current is between about 0.06 microamperes and about 6 milliamperes at 100% faradaic efficiency; and the preferred current at 100% faradaic efficiency is between about 0.6 microamperes and about 600 microamperes. At a practical faradaic efficiency of about 15%, the preferred current is at least about 4 microamperes and is not more than about 4 milliamperes.

Oxidation Catalysts and Reduction Catalysts

Use of catalysts of electrochemical oxidation reactions, also referred to as electrooxidation catalysts, is advantageous both in AC and in DC powered pain-relieving systems. In the AC systems, the electrooxidation catalyst is applied to all electrodes. In DC operation it is applied to the anode, also termed here the first electrode.

In DC operation, which is faradaically usually more efficient in generating the oxidant or oxidant precursor, the electrochemical generation of the desired pain-relieving oxidant or oxidant-precursor at a first electrode, an anode, is generally accompanied by generation of a hydrated proton, and an undesired acidic environment may be created. Near the second electrode, a cathode, an undesired basic environment may be created. The formation of strongly basic or strongly acidic cytotoxic regions can be avoided by passing and alternating current, AC. Upon passage of an AC-current strong acid generated in the anodic half-cycle is neutralized by strong base generated in the cathodic half-cycle. However, the faradaic yield of the pain-relieving oxidant, preferably a glutathione (GSH) oxidant or its precursor, is usually low when an alternating current is passed, because oxidant or oxidant-precursor electrosynthesized in the anodic-half cycle, is electroreduced in the cathodic half of the cycle. Achievement of a high faradaic yield of the oxidant generally requires that at a substantial part of the electrochemically synthesized pain-relieving oxidant not be subsequently electroreduced.

Advantageous operation of the pain relieving oxidant or oxidant-precursor synthesizing cell, without excessive cell-damage or cell-killing and at high faradaic efficiency is achieved when a DC current is passed between a compositionally different electrode pair, consisting of two electrodes with differing electrocatalysts. Here the electrocatalyst of a first electrode of a pair, which is the anode when a DC current is passed, differs from that of the electrocatalyst of the second electrode, which is the cathode of the pair. The electrocatalyst of the first electrode of the pair catalyzes the reaction whereby the pain relieving oxidant, or precursor oxidant, is electrochemically synthesized. The electrocatalyst of the second electrode of the pair catalyzes the electroreduction of water, and/or the electroreduction of dissolved oxygen, and/or both. The first electrode electrocatalyst catalyzes the electrochemical synthesis of the oxidant or its precursor, for example by the reaction of Equation 1; the second electrode electrocatalyst catalyzes the electrochemical reduction of water (Equation 2) and/or of oxygen (Equations 3 and/or 4).

The first electrode electrocatalyst of DC electrolysis, also referred to as the anode electrocatalyst in the context of DC electrolysis, is generally chosen from the group of electrocatalysts used in the electrooxidation of the aqueous chloride anion to chlorine, or to hypochlorous acid, or to hypochlorite anion. The first electrode or anode electrocatalysts include, for example, oxides of ruthenium and/or of iridium, of which ruthenium dioxide and/or iridium dioxide is generally preferred. Optionally, the electrodes comprising ruthenium oxide and/or iridium oxide electrocatalysts, have an electron or hole-conducting base, such as a metallic sheet or film, preferably comprising titanium and/or niobium and/or tantalum.

The second electrode-electrocatalyst, also referred to as the cathode electrocatalyst in the context of DC electrolysis, is chosen from the group of electrocatalysts known to be used for the electroreduction of water to hydrogen, or of oxygen to hydrogen peroxide and/or to water. Such electrocatalysts comprise, for example, platinum and/or palladium; copper; cobalt, iron or other transition metal-comprising porphyrins or phthalocyanins; polyoxometalates of molybdenum and/or tungsten; silver; gold, optionally combined with a quinone; an enzyme such as a multicopper oxidase enzyme, such as bilirubin oxidase. The base-conductor of the second electrode or cathode can comprise, for example, graphite, or small particles or fibers of carbon. The second electrode or cathode may alternatively comprise an electroreducible reactant. The electroreducible reactant, which need not be an electrocatalyst, can be, for example, silver chloride of a silver/silver chloride electrode, or nickel oxide of a nickel/ nickel oxide electrode.

Optionally, the DC electrochemical cell implanted in the body of a patient, in which a pain-reducing oxidant and/or its precursor is electrochemically synthesized, comprises body fluid as the electrolyte; a first electrode with an electrocatalyst for the electrooxidation of chloride of the body fluid; a second electrode with an electrocatalyst for the electroreduction of water and/or oxygen in the body fluid; and optionally a membrane between the first electrode and the second electrode, which is preferably an ion-conducting membrane, exemplified by a charge mosaic membrane, which is more permeable to both cations and anions than it is to non-ionic solution species. Optionally, the AC electrochemical cell implanted in the body of a patient in which a pain-reducing oxidant and/or its precursor is electrochemically synthesized comprises body fluid as its electrolyte; and electrodes, some or all of which are coated with an electrocatalyst for the electrooxidation of chloride of the body fluid.

Voltage

The voltage between the electrode-side terminals of the leads to which the electrodes are connected exceeds the thermodynamic potential required for the electrolysis in which the pain relieving oxidant or its precursor is generated. It is, typically, at least about 0.6 V, when the current flowing through the chloride electrooxidizing anode is greater than about 1 mA cm⁻². Because at high voltages and high current rapid electrolysis of water can produce a large volume of gaseous oxygen and hydrogen, it is preferred that the voltage between the terminals be less than about 2 V.

Types of Current

The current passed is generally a direct current, a pulsatile direct current, a rectified AC current, or a direct current with an alternating component. When an AC current is used, the direction of the current is switched generally fewer than about 100 times per second, preferably fewer than about 10 times per second, more preferably fewer than about 1 times per second and most preferably fewer than 0.1 times per second.

Ion Conducting Membranes

Strongly acidic or strongly basic environments are toxic to cells of a patient. When a DC current is passed, the environment near an operating anode can be strongly acidic, and near an operating cathode, it can be strongly basic. Because it is desirable to prevent the death of many cells in the process of the synthesis of the pain-relieving oxidant, it is preferred that the faradaic yield of the oxidant be high and there be as little as possible accumulation of either a strong acid or of a strong base near an electrode. This is accomplished by neutralizing the acid by the base, or neutralizing by the body fluid's own buffer. In the electrochemical synthesis of the very weak and therefore acidity-wise innocuous hypochlorous acid by the catalyzed electrooxidation of chloride at a first electrode or in DC operation an anode (Equation 1), a strong acid, represented in the equation as a proton, is co-generated. At the second electrode, or in DC operation a cathode, the strong base OH— is often co-generated, for example when water is catalytically electroreduced to hydrogen, (Equation 2), or when dissolved oxygen is catalytically electroreduced to either water (Equation 3), or to hydrogen peroxide (Equation 4).

Cl³¹+H₂O→HClO+2e⁻+H⁺ (1)

2H₂O+2e⁻→H₂+2OH⁻ (2)

O₂+4e⁻+2H₂O→H₂O+4OH⁻ (3)

O₂+2e⁻+2H₂O→H₂O₂+2OH⁻ (4)

In order to substantially shorten the half-lives of the co-generated strong acids and bases, but not of the electrochemically generated oxidant, the preferred electrolytic cells allow rapid transport of positively charged ions or negatively charged ions from the proximity of the anode(s), to the cathode(s) and/or rapid transport of ions from the cathode(s) to the anode(s), in order to neutralize the acid and the base, without allowing the rapid flow of the less ionized or non-ionic or zwitterionic neutral oxidant generated at the anode to the cathode, where it could be electroreduced. The membrane is less permeable to uncharged, or zwitterionic molecules, such as oxidizing agents, than it is to largely dissociated (and thus charged) strong acids and/or strong bases. A membrane can be selected such that ionically dissociated, strong, less than about 200 dalton mass, strong acids and/or strong bases permeate through the membrane at least twice as rapidly as less than about 200 dalton mass uncharged or zwitterionic oxidizing agents. This can be accomplished, for example, by inserting between the anode and the cathode a charge mosaic membrane, which is at least about twice less permeable to a non-ionic or a zwitterionic oxidant of a mass of less than 200 dalton than it is to a less than 200 dalton mass strong acid and/or a strong base. Ionically dissociated, strong, less than about 200 dalton mass, strong acids and/or strong bases permeate through the membrane at least twice as rapidly as less than about 200 dalton mass uncharged or zwitterionic oxidizing agents. Preferably the membrane is at least tenfold more permeable ionically dissociated, strong, less than about 200 dalton mass, strong acids and/or strong bases than it is to less than about 200 dalton mass, uncharged or zwitterionic, oxidizing agents. Such a membrane may comprise, for example, a charge mosaic membrane. The location of the charge mosaic membrane can be anywhere between the anode and a cathode of the electrochemical cell implanted in a patient; preferably, it is closer to the second electrode, the cathode, than it is to the first electrode, the anode, and most preferably it is proximal to or in contact with the second electrode, the cathode. Thus, it can be applied to the cathode, for example, by dipping the cathode in the polymer solution, by spraying the cathode with the polymer solution, by painting or brushing or spraying the polymer solution on the cathode, by doctor blading a paste of the polymer on the cathode. Usually the application of the polymer solution onto the cathode is followed by solvent evaporation and curing steps.

Ion conducting membranes that can be used in accordance with the devices of the invention include those described in U.S. Pat. Nos. 4,284,492; 4,514,304; 4,976,860; 5,543,045; 6,472,479; 6,484,887; and 6,663,775, each of which is incorporated herein by reference.

The pain relieving oxidant or oxidant-precursor producing cell is typically, but not necessarily, implanted in the body of a patient. It includes, in DC operation, one or multiple sets of optionally paired electrodes, a first electrode or anode at which the pain relieving oxidant, or its oxidant precursor, is generated and a second electrode or cathode at which reduction takes place. The electrolytic solution of the cell is usually fluid of the tissue in which the cell is implanted, filtered optionally by a filter the bio-fouling of which is reduced by a coating such as a polyethylene glycol comprising coating. In a preferred embodiment, the first and second electrodes of the paired electrodes are separated by an ion conducting membrane, such as a charge mosaic membrane, which is preferably proximal to, and/or is adhered to the second electrode, the cathode when the current passed is a direct current. The spacing between a paired anode and its preferably compositionally different paired cathode is small enough for diffusion of the anode-generated strong acid to the cathode, where it combines with and neutralizes cathode-generated strong base, the neutralization minimizing the volume in which cells may die because of low or high pH. It is generally preferred that the distance between the paired anode and cathode be less than about 5 mm, more preferably less than about 3 mm and most preferably less than about 1 mm. It is also preferred that the both positively charged ion conducting and negatively charged ion conducting membrane, which retards the transport of less-ionic species, non-ionic molecules or zwitterions, be thinner than about 1 mm, more preferably thinner than about 0.5 mm and most preferably thinner than about 0.2 mm.

Charge Mosaic Membranes

Suitable charge-mosaic membranes may be prepared using numerous methods well known in the art. For example, cation exchange resins may be combined with anion exchange resins using a polystyrene binder (see e.g., U.S. Pat. No. 2,987,472) or a silicone resin (see e.g., J. N. Weinstein et al., Desalination 12:1(1973)). Alternatively, suitable membranes may also be fabricated by casting or blending polymer phases (see e.g., Shorr et al., Desalination 14:11(1974) or Japanese Laid-Open Specification No. 14389/1979). Still further suitable methods include ionotropic-gel membrane methods (see e.g., H. J. Purz, J. Polym. Sci., Part C 38:405(1972)), latex-polymer electrolyte methods (see e.g., Japanese Laid-Open Specification No. 18482/1978), or block copolymerization methods (see e.g., Y. Isono et al., Macromolecules 16:1(1983)). In yet further contemplated methods, a cationic, anionic, or neutral polymer may be derivatized to include positive and negative charges suitable for ion exchange.

Where cationic and anionic polymers are employed to form a charge mosaic membrane, cationic polymers preferably include primary, secondary or tertiary amino groups, quaternary ammonium groups, or salts thereof, while anionic polymers preferably include sulfonic groups, carboxylic groups or salts thereof. Suitable cationic polymers include polyvinylpyridine and quaternized products thereof; poly(2-hydroxy-3-methacryloyloxypropyltrimethylammonium chloride); poly(dimethylaminoethyl methacrylate), poly(diethylaminoethyl methacrylate), and copolymers with other monomers and/or polymers. Suitable anionic polymers include poly-(2-acryloylamino-2-methyl-1-propanesulfonic acid), poly(2-acryloylamino-2-propanesulfonic acid), polymethacryloyloxypropylsulfonic acid, polysulfopropyl methacrylate, poly(2-sulfoethyl methacrylate), polvinylsulfonic acid, polyacrylic acid, polystyrene-maleic acid copolymers, and copolymers with other monomers and/or polymers.

Furthermore, at least one of the cationic and anionic polymers may be crosslinked using crosslinkers well known in the art. Among numerous alternative crosslinkers, contemplated crosslinkers include divinylbenzene, methylenebisacrylamide, ethylene glycol dimethacrylate and 1,3-butylene glycol dimethacrylate as well as tri- or tetra-functional acrylates and methacrylates. Still further contemplated charge mosaic membranes include those described in U.S. Pat. Nos. 4,976,860 and 5,304,307, each of which is incorporated herein by reference.

Use of Electrode Arrays

Arrays of multiple electrodes can be used for pain relief. The use of arrays is advantageous for example when only the approximate position of the pain-sensing nerve or the pain-transmitting nerve or synapse is known. In this case the likelihood of having at least one electrode near the source of pain is increased by using an array comprising multiple electrodes on which the oxidant or its precursor is electrosynthesized, preferably by an electrocatalyzed reaction, and most preferably by the catalyzed electrooxidation of chloride.

Coating Methods

Methods for incorporating electrooxidation catalysts and/or electroreduction catalysts onto or into the electrodes of the invention include: (a) directly affixing to the lead and/or the electrode an electrocatalyst (e.g., by either a spraying process or dipping process, with or without a carrier); (b) directly incorporating into the lead and/or the electrode an electrocatalyst (e.g., by either a spraying process or dipping process as described above, with or without a carrier); (c) by coating the lead and/or the electrode with a substance such as a hydrogel which may in turn absorb the an electrocatalyst; (d) by inserting the lead and/or the electrode into a sleeve or mesh which is comprised of, or coated with, an electrocatalyst; (e) constructing the lead and/or the electrode itself (or a portion of the device and/or the electrode) with an electrocatalyst; (f) by covalently binding the electrocatalyst directly to the lead and/or electrode surface or to a linker (small molecule or polymer) that is coated or attached to the device surface; (g) electrodepositing the electrocatalyst on the lead and/or electrode surface; (h) evaporating or sputtering the electrocatalyst on the lead and/or the electrode surface (i) chemically vapor depositing the electrocatalyst on the lead and/or the electrode surface; (j) painting the electrocatalyst on the lead and/or the electrode surface; or (k) doctor blading a film of the electrocatalyst precursor on the lead and/or electrode surface. Any of these deposition processes may be optionally followed by a heating, baking or firing step. Each of these methods illustrates an approach for converting a neurostimulation device to an electrolytic device with an electrocatalyst according to the present invention.

For these devices, leads and electrodes, the coating process can be performed in such a manner as to: (a) coat the non-electrode portions of the lead or device; (b) coat the electrode portion of the lead; or (c) coat all or parts of the entire device with the electrocatalyst. Additionally, or alternatively, the electrocatalyst can be mixed with the materials that are used to make the device, lead and/or electrode such that the electrocatalyst and/or oxidizable agent is incorporated into the final product. In these manners, a medical device may be prepared which has a coating, where the coating is, e.g., uniform, non-uniform, continuous, discontinuous, or patterned.

External and Internal Reservoirs Containing an Oxidizable Agent.

The electrochemically generated oxidant, e.g., hypochlorous acid, can be reacted with an agent controllably released from an implant proximal to the electrode. It can alternatively be infused using a connecting cannula to the proximity of the electrode at which the oxidant is electrochemically generated from an implanted reservoir, for example a refillable reservoir. The implanted reservoir can be refilled, for example, by injecting into it a solution using a syringe with a needle. Alternatively the solution of the agent can be infused from an external reservoir, for example a skin attached refillable or non-refillable reservoir. The infusion to the proximity of the electrochemically oxidant generating electrode can be through a connecting cannula. Alternatively, the solution containing the agent can be injected into the tissue proximal to the electrode at the site the oxidant is generated. Rapid reaction of the electrochemically generated oxidant with the controllably released or infused or injected solution of the agent is particularly advantageous when an AC current is passed, because some or all of the oxidant, such as hypochlorous acid, generated in the oxidizing half cycle, can react with the agent before the direction of the current is reversed, reducing the likelihood of electroreduction of the electrochemically generated oxidant and increasing the yield of a pain relieving oxidant compound or of its precursor. To allow enough time for the electrochemically generated oxidant to react with the controllably released or injected or infused agent, the frequency of the AC is generally less than about 100 Hz, is preferably less than 10 about Hz is more preferably less than about 1 Hz and is most preferably less than about 0.1 Hz. Reaction of the electrochemically produced oxidant with the controllably released or infused or injected agent may also provide, in DC or in AC operation, a longer lived oxidant and/or an oxidant that is more selective in its pain relieving reaction with the targeted component of the neural tissue.

Oxidizable Agents

The preferred oxidizable agent released from the implanted reservoir, or infused from the internal or external reservoir, or injected from an external reservoir can be an ammonium salt, reacting to form pain-relieving chloramine; or taurine, reacting to form N-chloro taurine, a natural chloramine known to be produced by leucocytes; or glutathione. It can also be a salt of an acid and a substituted amine, a thiol, or a thiolate salt. It can be, for example, urea, oxidized glutathione, glutathione sulfonamide, glycine, sulfamic acid, sarcosine, alpha-aminoisobutyric acid, acetylglycine, alanine, beta-alanine, phenyl alanine, norvaline, leucine, isoleucine, proline, omega aminoundecanoic acid, spartic acid, glutamic acid, asparagine, valine, tyrosine, threonine, cysteine, cystine, methionine, glutamine, tryptophane, histidine, arginine, lysine, alpha-aminobutyric acid, gamma-aminobutyric acid, alpha, epsilon diamino pimelic acid, ornithine, anthranilic acid, p-aminobenzoic acid, sulfanilic acid, orthanilic acid, phenyl sulfamic acid, aminopropanesulfonic acid, ethylenediamine tetraacetic acid, aminomethane-sulfonic acid, glycylglycine, glycylglycylglycine, metanilic acid, methylamine, ethylamine, and N-octodecanyl glycine; or a thiol-comprising peptide.

Use of a Saline Solution

In one approach for the treatment of pain, a solution is infused from an external reservoir through a cannula to the proximity of the treated nerve ending or nerve. The reservoir contains about isotonic, about 0.15 M NaCl, saline, buffered, for example by about 20 mM phosphate buffer, to about neutral pH and includes at least one anode and at least one cathode connected through leads to a DC power supply, preferably a battery. The oxidizable agent is dissolved in the saline at a typical concentration between about 0.01 mM and about 3 mM. The anode is optionally coated with an electrocatalyst such as ruthenium oxide or iridium oxide, or it can be graphite, without a catalyst. The cathode can include a water or oxygen reduction catalyst. The electrochemically generated oxidant, e.g., hypochlorous acid, reacts with the dissolved oxidizable agent to produce the pain-relieving precursor or its oxidant. Anodically generated acid is neutralized by cathodically generated base. The solution in the reservoir can be controllably infused using a connecting cannula to the proximity of the treated nerve. In certain embodiments, the reservoir is adhered to the skin and/or is refillable. For safety, it is preferred that the total amount of pain relieving oxidant or oxidant precursor contained in the reservoir be such that its accidental release would not be harmful.

In a related approach for the treatment of pain, the saline solution and oxidizable agent are contained within an implanted reservoir in which the oxidant or its precursor is electrochemically generated.

In still another approach, the saline solution and oxidizable agent are contained in an external reservoir or implanted reservoir and are released in the proximity of an implanted electrode. The solution in the reservoir is controllably infused using a connecting cannula to the proximity of the operating electrode. The electrode can be an anode of a DC powered implanted cell or it can be one of the electrodes of an AC powered cell.

Alternatively, the solution containing the oxidizable agent can be injected into the tissue proximal to the electrode at the site the oxidant is generated. Rapid reaction of the electrochemically generated oxidant with the controllably released or infused or injected solution of the agent is particularly advantageous when an AC current is passed, because some or all of the oxidant, such as hypochlorous acid, generated in the oxidizing half cycle, can react with the agent before the direction of the current is reversed, reducing the likelihood of electroreduction of the electrochemically generated oxidant and increasing the yield of a pain relieving oxidant compound or of its precursor. To allow enough time for the electrochemically generated oxidant to react with the controllably released or injected or infused agent, the frequency of the AC is generally less than about 100 Hz, 10 Hz, 1 Hz, or 0.1 Hz.

Alternatively, the oxidizable agent can be controllably released, for example from a dissolving or biodegrading polymer. Reaction of the electrochemically produced oxidant with the controllably released oxidizable agent may provide, in DC or in AC operation, a longer lived oxidant and/or an oxidant that is more selective in its pain relieving reaction with the targeted component of the neural tissue.

### Non-Electrochemical Synthesis of Hypochlorous Acid

The methods and devices of the invention also include non-electrochemical synthesis of hypochlorous acid in an implant or in a wound-fluid exposed dressing for the treatment of pain and itch. Myeloperoxidase-catalyzed oxidation of chloride anion by hydrogen peroxide to hypochlorous acid (Reaction 5) is known to take place in inflamed tissues. Both stimulated neutrophils and macrophages express myeloperoxidases and produce hypochlorite.

Cl—+H₂O₂→ClO—+H₂O (5)

Pain relieving amounts of hypochlorous acid can be produced enzymatically in the body (e.g., as part of an implantable system) or in a wound-fluid exposed dressing in two steps. In the first step, hydrogen peroxide would be generated, for example by glucose oxidase catalyzed oxidation of body fluid glucose by body fluid oxygen, or by lactate oxidase catalyzed oxidation of body fluid lactate by body fluid oxygen (see Reactions 6 and 7). In the second step, hypochlorous acid would be generated by myeloperoxidase-catalyzed oxidation of body fluid chloride by the hydrogen peroxide generated in the first step (see Reaction 5).

Glucose+O₂→gluconolactone+H₂O₂ (6)

Lactate+O₂→pyruvate+H₂O₂ (7)

The spontaneous reaction of any ammonium salt, e.g. of ammonium chloride, with hypochlorous acid, yields pain relieving chloramine (Reaction 8):

NH₄⁺+HOCl→NH₂Cl+H₂O+H⁺ (8)

To generate chloramine by reacting of electrochemically or enzymatically produced hypochlorous acid, one can add, for example, ammonium carbonate to the wound dressings, or adsorb ammonium polystyrene sulfonate in fibers of the integrated wound dressings.

The implants can contain, for example, co-immobilized and sol-gel stabilized glucose oxidase and myeloperoxidase, or lactate oxidase and myeloperoxidase (their substrates are glucose and lactate, respectively) as a high-surface are aerogel. The product of the two-step reaction is the very weak hypochlorous acid, only partly ionized at pH 7.3 to the hypochlorite anion. The implant could be enclosed, for example, in a tissue-compatible hydrogel, such as a crosslinked poly(ethylene glycol) hydrogel, which is permeable to glucose, lactate, pyruvate, oxygen and other water-soluble species necessary to complete the reaction.

In wound dressings glucose oxidase and myeloperoxidase, or lactate oxidase and myeloperoxidase could be co-immobilized, for example, in an aerogel. The oxidase-catalyzed O₂-oxidation of wound-fluid glucose or lactate produces hydrogen peroxide, which oxidizes chloride to hypochlorous acid in the myeloperoxidase-catalyzed reaction. The dressing may also contain an oxidizable agent (e.g., any oxidizable agent described herein), such as ammonium carbonate, ammonium polystyrene sulfonate, taurine, or glutathione.

### Indications

The methods and devices of the invention are useful for treating pain, including clinical pain, namely inflammatory pain, functional pain, nociceptive pain, and neuropathic pain (e.g., peripheral neuropathic pain), whether acute or chronic (e.g., pain lasting for greater than one, two, three, four, or more months). Conditions that may be associated with pain include, for example, soft tissue, joint, bone inflammation and/or damage (e.g., acute trauma, osteoarthritis, or rheumatoid arthritis), myofascial pain syndromes (fibromylagia), stump pain, myocardial infarction, angina, ischemic cardiovascular disease, post-stroke pain, sickle cell anemia, peripheral vascular occlusive disease, cancer, inflammatory conditions of the skin or joints, diabetic neuropathy, and acute tissue damage from surgery or traumatic injury (e.g., lacerations or fractures). The present invention is also useful for the treatment, reduction, or prevention of musculo-skeletal pain (after trauma or exercise), neuropathic pain caused by spinal cord injury, tumors, compression, inflammation, dental pain, episiotomy pain, deep and visceral pain (e.g., heart pain, bladder pain, or pelvic organ pain), muscle pain, eye pain, orofacial pain (e.g., odontalgia, trigeminal neuralgia, glossopharyngeal neuralgia), abdominal pain, gynecological pain (e.g., dysmenorrhea and labor pain), pain associated with nerve and root damage due to trauma, compression, inflammation, toxic chemicals, metabolic disorders, hereditary conditions, tumors, infections, demyelinating diseases including multiple sclerosis, chronic lower back pain (e.g., ankylosing spondylitis, degenerative disk disease, radiculopathy, and radicular pain), sciatica, chronic neck pain, and post-operative pain (e.g., mastectomy, orthopedic and phantom limb pain). The present invention is also useful for treating pain associated with post-herpetic neuralgia, cancer, cystic fibrosis, HIV, and polymyalgia rheumatica. The methods and devices of the invention can be used to treat pain associated with any of a number of conditions, including back and neck pain, cancer pain, gynecological and labor pain, arthritis and other rheumatological pains, orthopedic pains, post herpetic neuralgia and other neuropathic pains, sickle cell crises, interstitial cystitis, urethritis and other urological pains, dental pain, postoperative pain, and procedural pain (i.e., pain associated with injections, draining an abcess, surgery, dental procedures, opthalmic procedures, arthroscopies and use of other medical instrumentation, cosmetic surgical procedures, dermatological procedures, setting fractures, biopsies, and the like).

Pain and Function Indices

In order to measure the efficacy of any of the methods or devices of the invention, a measurement index may be used. Indices that are useful in the methods and devices of the invention for the measurement of pain associated with musculoskeletal, immunoinflammatory and neuropathic disorders include a visual analog scale (VAS), a Likert scale, categorical pain scales, descriptors, the Lequesne index, the WOMAC index, and the AUSCAN index, each of which is well known in the art. Such indices may be used to measure pain, itch, function, stiffness, or other variables.

A visual analog scale (VAS) provides a measure of a one-dimensional quantity. A VAS generally utilizes a representation of distance, such as a picture of a line with hash marks drawn at regular distance intervals, e.g., ten 1-cm intervals. For example, a patient can be asked to rank a sensation of pain or itch by choosing the spot on the line that best corresponds to the sensation of pain or itch, where one end of the line corresponds to “no pain” (score of 0 cm) or “no itch” and the other end of the line corresponds to “unbearable pain” or “unbearable itch” (score of 10 cm). This procedure provides a simple and rapid approach to obtaining quantitative information about how the patient is experiencing pain or itch. VAS scales and their use are described, e.g., in U.S. Pat. Nos. 6,709,406 and 6,432,937.

A Likert scale similarly provides a measure of a one-dimensional quantity. Generally, a Likert scale has discrete integer values ranging from a low value (e.g., 0, meaning no pain) to a high value (e.g., 7, meaning extreme pain). A patient experiencing pain is asked to choose a number between the low value and the high value to represent the degree of pain experienced. Likert scales and their use are described, e.g., in U.S. Pat. Nos. 6,623,040 and 6,766,319.

The Lequesne index and the Western Ontario and McMaster Universities (WOMAC) osteoarthritis index assess pain, function, and stiffness in the knee and hip of OA patients using self-administered questionnaires. Both knee and hip are encompassed by the WOMAC, whereas there is one Lequesne questionnaire for the knee and a separate one for the hip. These questionnaires are useful because they contain more information content in comparison with VAS or Likert. Both the WOMAC index and the Lequesne index questionnaires have been extensively validated in OA, including in surgical settings (e.g., knee and hip arthroplasty). Their metric characteristics do not differ significantly.

The AUSCAN (Australian-Canadian hand arthritis) index employs a valid, reliable, and responsive patient self-reported questionnaire. In one instance, this questionnaire contains 15 questions within three dimensions (Pain, 5 questions; Stiffness, 1 question; and Physical function, 9 questions). An AUSCAN index may utilize, e.g., a Likert or a VAS scale.

Indices that are useful in the methods and devices of the invention for the measurement of pain include the Pain Descriptor Scale (PDS), the Visual Analog Scale (VAS), the Verbal Descriptor Scales (VDS), the Numeric Pain Intensity Scale (NPIS), the Neuropathic Pain Scale (NPS), the Neuropathic Pain Symptom Inventory (NPSI), the Present Pain Inventory (PPI), the Geriatric Pain Measure (GPM), the McGill Pain Questionnaire (MPQ), mean pain intensity (Descriptor Differential Scale), numeric pain scale (NPS) global evaluation score (GES) the Short-Form McGill Pain Questionnaire, the Minnesota Multiphasic Personality Inventory, the Pain Profile and Multidimensional Pain Inventory, the Child Heath Questionnaire, and the Child Assessment Questionnaire.

Itch can be measured by subjective measures (VAS, Lickert, descriptors). Another approach is to measure scratch which is an objective correlate of itch using a vibration transducer or movement-sensitive meters.

### OTHER EMBODIMENTS

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. An implantable medical device for the treatment of pain comprising a DC power supply in electrical communication with a first electrode and a second electrode, wherein said first electrode comprises an oxidation catalyst and said second electrode comprises either no catalyst, a reduction catalyst, or a reducible metal salt or metal oxide.

2. An implantable medical device for the treatment of pain comprising a DC power supply in electrical communication with a first electrode and a second electrode, wherein said first electrode comprises either no catalyst or an oxidation catalyst and said second electrode comprises a reduction catalyst or a reducible metal salt or metal oxide.

3. The implantable medical device of claims 1 or 2, wherein said first electrode comprises an oxidation catalyst and said second electrode comprises a reduction catalyst or a reducible metal salt or metal oxide.

4. The implantable medical device of claims 1 or 2, wherein said first electrode comprises an oxidation catalyst for catalyzing the electrooxidation of chloride anion.

5. The implantable medical device of claim 4, wherein said oxidation catalyst comprises an oxide of ruthenium or an oxide of iridium.

6. The implantable medical device of claim 5, wherein said oxidation catalyst is coated on an underlayer of an oxide of titanium metal or an oxide of tantalum.

7. The implantable medical device of claim 5, wherein said oxidation catalyst wherein said oxidation catalyst comprises ruthenium dioxide.

8. The implantable medical device of claim 7, wherein said oxidation catalyst comprises iridium dioxide.

9. The implantable medical device of claims 1 or 2, wherein said second electrode comprises a reducible metal salt or metal oxide.

10. The implantable medical device of claim 9, wherein said reducible metal salt or metal oxide is a silver salt or nickel oxide.

11. The implantable medical device of claim 10, wherein said reducible metal salt or metal oxide is silver chloride.

12. The implantable medical device of claims 1 or 2, wherein said second electrode comprises a reduction catalyst selected from platinum, palladium, silver, gold, copper, a porphyrin-metal complex, a phthalocyanin-metal complex, a polyoxometalate of molybdenum, a polyoxometalate of tungsten, a quinone, or a multicopper oxidase enzyme.

13. The implantable medical device of claims 1 or 2, wherein said first electrode is an anode comprising graphite.

14. The implantable medical device of claims 1 or 2, wherein said first electrode and said second electrode are separated by an ion conducting membrane.

15. The implantable medical device of claim 14, wherein said ion conducting membrane conducts both anions and cations.

16. The implantable medical device of claims 1 or 2, wherein said implantable medical device when implanted in a patient generates hypochlorous acid in an amount sufficient to treat pain.

17. An implantable medical device for the treatment of pain comprising a DC power supply in electrical communication with a first electrode and a second electrode, wherein said first electrode and said second electrode are separated by an ion conducting membrane.

18. The implantable medical device of claim 17, wherein said first electrode comprises an oxidation catalyst or said second electrode comprises a reduction catalyst or a reducible metal salt or metal oxide.

19. The implantable medical device of claim 18, wherein said ion conducting membrane conducts both anions and cations.

20. The implantable medical device of claim 19, wherein said ion conducting membrane is a charge mosaic membrane.

21. An implantable medical device for the treatment of pain comprising an AC power supply in electrical communication with a first electrode and a second electrode, wherein each of said first electrode and said second electrode comprise an oxidation catalyst.

22. The implantable medical device of claim 21, wherein said oxidation catalyst comprises an oxide of ruthenium or an oxide of iridium.

23. The implantable medical device of claim 21, wherein said AC power supply has a frequency of less than 100 Hz.

24. The implantable medical device of claim 17 or 21, wherein said implantable medical device when implanted in a patient generates hypochlorous acid in an amount sufficient to treat pain.

25. The implantable medical device of claims 1, 2, 17, or 21, further comprising a reservoir in fluid communication with said first electrode, said reservoir comprising an oxidizable agent selected from amines, amides, thiols, and salts thereof.

26. The implantable medical device of claim 25, wherein said reservoir further comprises a chloride salt.

27. An implantable medical device for the treatment of pain comprising (i) a power supply in electrical communication with a first electrode and a second electrode, wherein an oxidant is electrochemically generated at least at the first electrode; (ii) a reservoir comprising a solution of an oxidizable agent in fluid communication with the electrochemically generated oxidant, wherein said oxidant oxidizes said oxidizable agent to produce an oxidized agent; and (iii) an exit port in fluid communication with said oxidized agent, wherein after implantation said implantable medical device generates oxidized agent in an amount sufficient to treat pain.

28. The implantable medical device of claim 27, wherein said oxidizable agent is selected from amines, amides, thiols, and salts thereof.

29. The implantable medical device of claim 28, wherein said oxidizable agent is selected from ammonia, taurine, glutathione, glutathione sulfonamide, and salts thereof.

30. The implantable medical device of claim 28, wherein said reservoir further comprises a chloride salt.

31. The implantable medical device of claim 27, wherein said reservoir is configured to be completely implanted within said patient.

32. The implantable medical device of claim 27, wherein said reservoir is configured to be positioned external to said patient and further comprising a cannula in fluid communication with said oxidant and said port, wherein said port is implanted within said patient.

33. The implantable medical device of claim 27, wherein said reservoir is configured to sit on the skin of said subject.

34. The implantable medical device of claim 27, wherein said reservoir is refillable.

35. The implantable medical device of claim 27, wherein said power supply is a DC power supply.

36. The implantable medical device of claim 27, wherein said power supply is an AC power supply.

37. The implantable medical device of claim 36, wherein said AC power supply has a frequency of less than 100 Hz.

38. The implantable medical device of any of claims 1, 2, or 17, wherein said power supply is operating at a voltage from 0.6 V to 5.0 V.

39. The implantable medical device of any of claims 1, 2, or 17, wherein said power supply is operating at a current density of less than 5 mA/cm².

40. A method of treating pain in a patient in need thereof, said method comprising (i) implanting an electrolytic device of any of claims 1-39 in said patient at the site of pain and (ii) operating said device to generate an oxidant in an amount sufficient to treat said pain.

41. The method of claim 40, wherein said pain is nociceptive pain, somatic pain, visceral pain, procedural pain, or inflammatory pain caused by trauma or surgery.

42. The method of claim 41, wherein said pain is caused by trauma, surgery, herniation of an intervertebral disk, spinal cord injury, shingles, HIV/AIDS, cancer related pain, amputation, carpal tunnel syndrome, diabetic neuropathy, postherpetic neuralgia, or a musculoskeletal disorder.

43. A biocompatible implantable matrix comprising (i) glucose oxidase or lactate oxidase; and (ii) myeloperoxidase.

44. The biocompatible implantable matrix of claim 43, wherein said matrix is a hydrogel.

45. The biocompatible implantable matrix of claim 43, further comprising an oxidizable agent is selected from amines, amides, thiols, and salts thereof.

46. A method of treating pain in a patient in need thereof, said method comprising implanting into said patient the biocompatible implantable matrix of any of claims 43-45.

47. The method of claim 46, wherein said biocompatible implantable matrix is implanted at the site of pain.
